(19) United States
(12) Patent Application Publication
(10) Pub. No.: US 2024/0216621 A1
(43) Pub. Date: Jul. 4, 2024

(54) SYSTEMS AND METHODS FOR INTELLIGENT VAPORIZERS

(71) Applicant: Zorday IP, LLC, Mayfield Village, OH (US)

(72) Inventor: Brent M. Skoda, Mayfield Village, OH (US)

(73) Assignee: Zorday IP, LLC, Mayfield Village, OH (US)

(21) Appl. No.: 18/232,999

(22) Filed: Aug. 11, 2023

Related U.S. Application Data
(63) Continuation of application No. 17/721,539, filed on Apr. 15, 2022, now abandoned.
Application No. 17/721,539 is a continuation of application No. 16/386,600, filed on Apr. 17, 2019, now abandoned.
Application No. 16/386,600 is a continuation of application No. 15/048,575, filed on Feb. 19, 2016, now abandoned.
(60) Provisional application No. 62/118,869, filed on Feb. 20, 2015.

(51) Int. Cl. A61M 11/04; A24F 40/10; A24F 40/30; A24F 40/42; A24F 40/65; A61M 15/00; A61M 15/06; A61M 16/00

(52) U.S. Cl. CPC A61M 11/042; A24F 40/30; A24F 40/42; A61M 15/0021; A61M 15/0065; A61M 15/06; A24F 40/10; A24F 40/65; A61M 15/0003; A61M 15/008; A61M 2016/0024; A61M 2016/0027; A61M 2205/3317; A61M 2205/3553; A61M 2205/3592; A61M 2205/502; A61M 2205/6018; A61M 2205/702

(57) ABSTRACT

A vaporization and inhalation apparatus comprising a cartridge container configured to receive a cartridge including a liquid. The apparatus includes a heating element configured to heat the liquid to a point of vaporization to generate a vaporized form of the liquid. The apparatus also includes an outlet port through which the vaporized form of the liquid is inhaled.

### RELATED APPLICATION

The present application is a continuation of and claims priority to U.S. patent application Ser. No. 17/721,539, titled “Systems and Methods for Intelligent Vaporizers” and filed Apr. 15, 2022, which is a continuation of and claims priority to U.S. patent application Ser. No. 16/386,600, titled “Systems and Methods for Intelligent Vaporizers” and filed Apr. 17, 2019, which is a continuation of and claims priority to U.S. patent application Ser. No. 15/048,575, titled “Systems and Methods for Intelligent Vaporizers” and filed Feb. 19, 2016 which in turn claims priority to and the benefit of U.S. Provisional Application No. 62/118,869, titled “Systems and Methods for Intelligent Vaporizers” and filed on Feb. 20, 2015, which are incorporated herein by reference in their entirety for all purposes.

### TECHNICAL FIELD

The present application relates generally to apparatuses and systems for operating vaporizers and inhalers.

### BACKGROUND

Vaporizers or inhalers may be used to administer, transform or otherwise dispense a substance in a consumable format (i.e. vapor, fine powder, mist, liquid) for the user. One form of vaporizers includes electronic cigarettes. The substance for consumption through a vaporizer or vaporization apparatus or device may include Caffeine, an energy boosting formulation, a flavored substance, a medicinal formula, a supplement, a vitamin, a mineral, any ingredient officially monographed and listed in the Homeopathic Pharmacopeia of the United States of America (the “HPUS”), or various other products for consumption alone or in combination. Vaporizers configured for consumption by a user via inhaling may be operated through the use of various electronic components configured to heat the substance, which substance may be stored as a liquid, to transform the liquid substance into a vapor phase and present the substance for consumption to the user via an outlet port configured to permit inhaling of the substance.

### SUMMARY OF THE INVENTION

Various embodiments disclosed herein provide apparatuses, systems, and methods related to the operation of a device configured to convert substances into a form configured for consumption by inhalation.

Various embodiments provide a vaporization and inhalation apparatus or device configured to vaporize a substance such as liquid contained in a cartridge through heating of the liquid to a point of vaporization. The device is further configured to permit a user to consume the vapor through inhaling via an outlet port in the device. In particular embodiments, the vaporization device includes at least one sensor device configured to determine a quantity of consumption from the cartridge based on the vapor draw. The device may include a counter and/or a timer configured to determine the number of vapor draws and or the duration of a vapor draw. The timer may be configured to determine the length of a vapor draw, for example based on a change in pressure in the device during consumption by the user. The timer may be configured to additionally or alternatively determine the duration of a heating event associated with the electronic heating or other heating of the substance to form a vapor in connection with a vapor draw request by a user. The device further includes at least one specially programmed processor configured to, based on the duration of the vapor draw and/or heating event associated with the vapor draw, determine a value associated with an estimate of a quantity consumed. The vapor draw may be based on predetermined aerosol tests. In particular embodiments, the specially programmed processor may determine power consumption to determine the duration of a vapor draw and/or a heating even to determine a value associated with an estimate of quantity consumed. The specially programmed processor may be configured to vary the consumption rate based on the sensed or measured power or time based on the substance in the cartridge. For example, the cartridge may include a unique identification code associated with particular substances. The processor may be calibrated such that the processor is configured to associate a particular consumption rate with particular substances to account for variations in heating rates for different substances. In various embodiments, the cartridge may be programmable and/or may include one or more memory device that may be communicably coupled with a processor in the vaporization device to provide identifying information such as substance contained therein and to provide historical information such number of prior uses, if any, remaining quantity on-board or initial quantity on-board, etc.

In various embodiments, the processor of the vaporization device may be configured to cause various actions based on the consumption determination. For example, the processor may be configured to prompt a refill request based on the quantity in the cartridge falling below a certain threshold, to replenish a supply to a user on time. The threshold may be pre-programmed, user specified, and/or may be automatically or manually adjustable based on user habits, locations, estimated time of delivery, or other aspects that may be programmed or learned through storage of various behavior aspects. In various embodiments, the vaporization device may include a location based system and/or may include a wireless transmitter configured to connect the device to one or more networks, such as the Internet. Accordingly, the vaporization device may, in response to prompting a refill request, determine the best mode of refilling and may transmit the request to a local distributer, including, but not limited to a pharmacy. The refill request may indicate a user's location, cartridge substance identifying information. The request may facilitate a refill cartridge be sent directly to the user at a pre-set location. The refill request may automatically send the refill to the closest distributor and/or may send the refill directly to the user. The vaporization device may store the quantity of refills consumed in the device, the date, or rate of refill requests for a user, the rate of consumption, the location of consumption, the time of consumption, dosage requirements, substance flavor when applicable, the substance consumed, and any other relevant information pertaining to the substance the user, the substance, or any relevant third party (i.e. doctor, pharmacist, manufacturer, distributor, storage temperatures, that would be relevant and known to one skilled in the art of whatever substance is being dispensed). Any of the aforementioned parameters may be stored separately or may be aggregated, for example to create a user profile and to enable machine learner and user preferences. The information may be used for advertising purposes and/or to offer discounts, deals on consumable products, or may be implemented in a social networking platform, for example to notify a consumer of others consuming similar substances at nearby locations for social consumption.

Various embodiments provide a vaporization device configured to vaporize a plurality of substances independently or collectively as selected. The plurality of substance includes a plurality of independently stored liquids, for example, contained in a single multi-content cartridge. The vaporization device may be configured to heat one or more of the liquids independently and to vary which one or more of the liquids is heated at any time to allow a user to switch substances for vaporization and or combine different vapors. The vaporization device may be specially programmed to heat different substances for different duration based on the substance. At least one of the device and or the multi-substance cartridge may be configured to identify or determine the substances provided in the cartridge and to permit a user selection of the substances for consumption through inhaling via an outlet port in the device.

Particular embodiments of the multi-substance cartridges may be configured to determine vapor draw quantities and user consumption for one or more of the substances contained in each section of the cartridge in accordance with various other embodiments described herein.

The inventors have appreciated that described embodiments disclosed herein provide vaporization and inhaler apparatuses that permit rapid and automated seamless refilling systems and multi-substance consumption in a compact and behavior assisted machine learning format.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

FIG. 1A is a block diagram depicting an embodiment of a network environment comprising client devices in communication with server devices.

FIG. 1B is a block diagram depicting a cloud computing environment comprising client devices in communication with a cloud service provider.

FIGS. 1C and 1D are block diagrams depicting embodiments of computing devices useful in connection with the methods and systems described herein.

FIG. 2 is a front view of a vaporization and inhaler apparatus according to an embodiment.

FIG. 3 is a flow diagram illustrating an operational regimen of the vaporization and inhaler apparatus of FIG. 2.

FIG. 4 is a front view of a vaporization and inhaler apparatus according to another embodiment.

FIG. 5 is a top cross-sectional view of the vaporization and inhaler apparatus of FIG. 4.

FIG. 6 is a flow diagram illustrating an operational regimen of the vaporization and inhaler apparatus of FIG. 4.

The features and advantages of the inventive concepts disclosed herein will become more apparent from the detailed description set forth below when taken in conjunction with the drawings.

### DETAILED DESCRIPTION

Following below are more detailed descriptions of various concepts related to, and embodiments of, inventive variable counterweight systems and methods of operating variable counterweight systems. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes a network environment and computing environment which may be useful for practicing various computing related embodiments described herein.

Section B describes embodiments of systems and methods facilitating consumption of a substance through a vaporization and inhaler apparatus.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### A. Computing and Network Environment

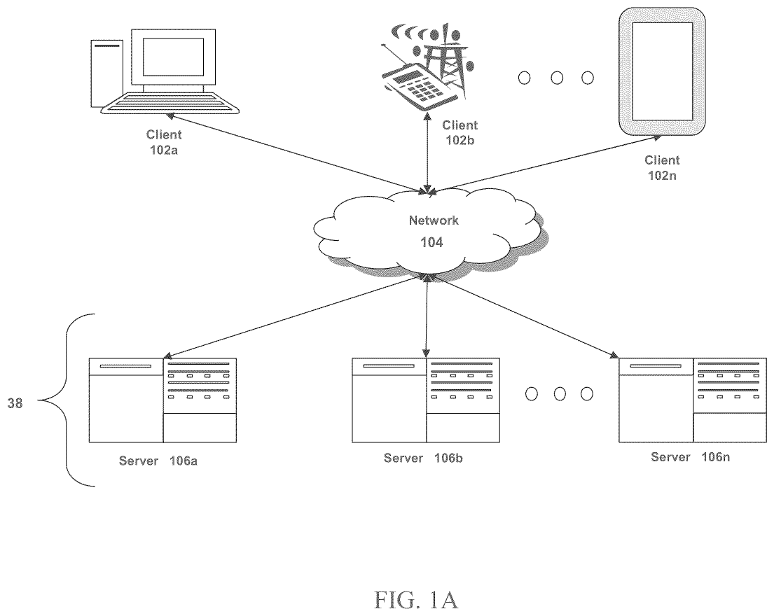

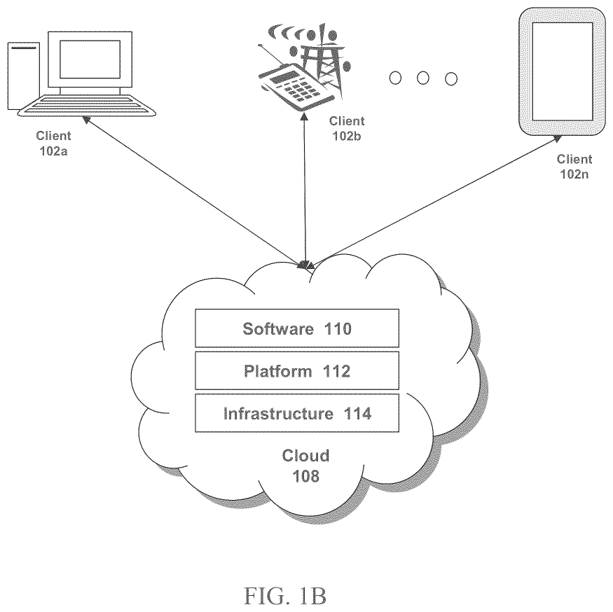

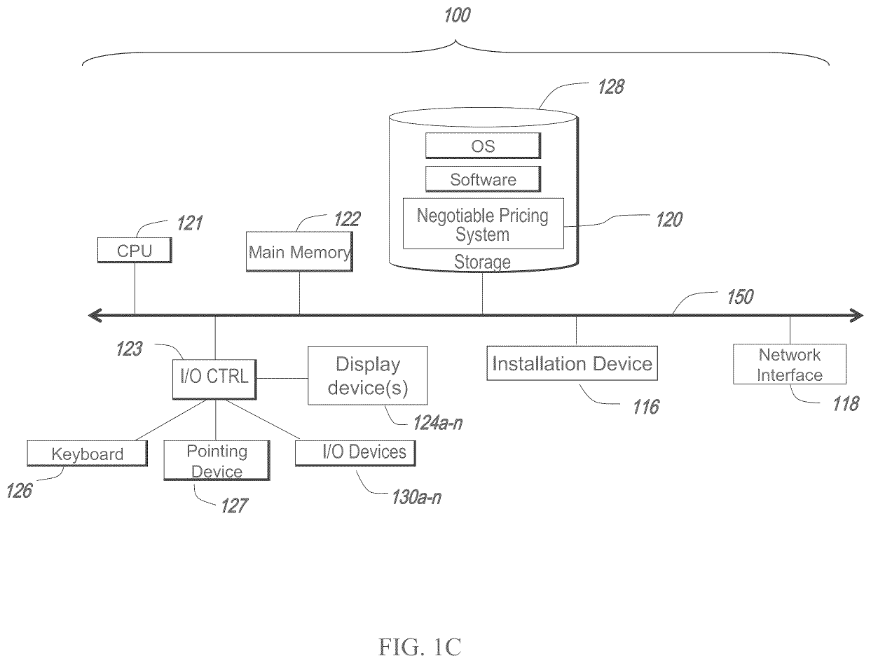

Prior to discussing specific inventive embodiments, it may be helpful to describe aspects of the operating environment as well as associated system components (e.g., hardware elements) in connection with the methods and systems described herein. Referring to FIG. 1A, an embodiment of a network environment is depicted. In brief overview, the illustrated exploring network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, NFC, RFID Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network, which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPV6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualized physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, and/or software of a vaporization system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software 120 for the vaporization system. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein.

In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call. In some embodiments, the communication device 102 is a wearable mobile computing device including but not limited to Google Glass and Samsung Gear.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the vaporization apparatus and related systems and methods disclosed herein.

### B. Systems and Methods of a Vaporization and Inhaler Apparatus

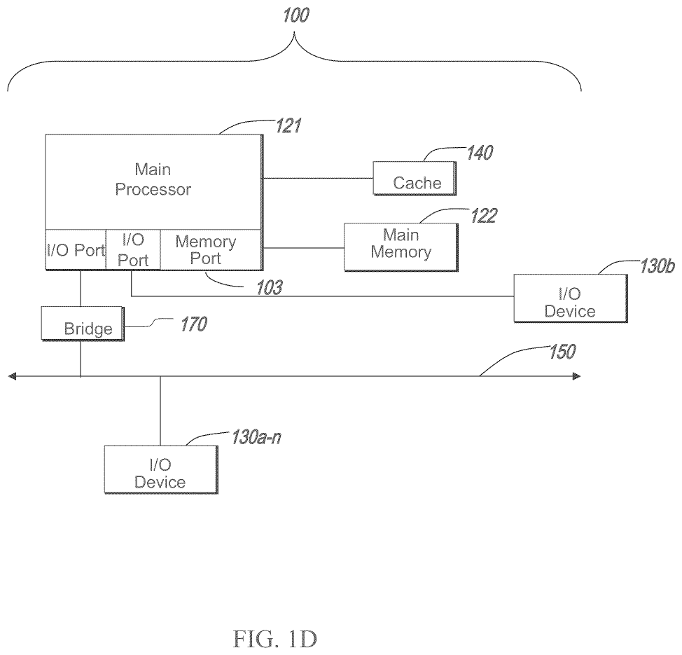

FIG. 2 is a front view of a vaporization apparatus and inhaler apparatus according to an embodiment. FIG. 2 provides a partial cross-sectional view in the vaporization and inhaler apparatus 201. The vaporization and inhaler apparatus 201 includes a substance cartridge 202, containing the substance, such as a liquid formulation, for vaporization and inhaling. In example embodiments, the substance cartridge 202 may include a solid formulation that may be crushed, pulverized, or atomized, or heated and vaporized for inhalation. The substance cartridge 201 may include a substance including, but not limited to caffeine, panax, ginseng, gingko, biloba, bitter orage, cola-nut, guarana, natrum carbonicum, green tea, cocoa extract, cannabis, yerba mate, other vaporizable or inhalable supplements, pharmaceuticals, medicines, waxes, or liquids. The substance cartridge 202 may include a cartridge identification code. The cartridge identification code may identify the substance contained in the cartridge, flavor identification, expiration information, or other pertinent information regarding the cartridge content. The cartridge identification code may be electronically stored in a programmable memory on board the substance cartridge 202, which programmable memory may include other information such as historical usage information, including but not limited to location of use, device of use, time of use, or other data associated with the cartridge. In example embodiments, the cartridge 202 may include an RFID tag associated with the cartridge for communication with the vaporization and inhaler apparatus 201 and or one or more other devices. The RFID tag may be an active or passive tag.

The vaporization and inhaler apparatus 201 also includes a control system 204 configured to determine usage information in connection with updating and monitoring the use of substances via the vaporization and inhaler apparatus 201, for example to assist with automated refilling in accordance with various embodiments disclosed herein. The control system 204 may include a processor 205 which may be specially programmed to determine quantity of a cartridge based on usage history and parameters. For example, aerosol test may be run on substance cartridge 202. The substance cartridge may include the information in the on-board memory or the information may be pre-programmed into the processor 205. The aerosol test may be based on a fully charged battery, such as battery 206, and a filled substance cartridge, test different voltages supplied, and determine how much power vaporizes how much of the given vaporizable or dispensable product from the cartridge based on a typical vapor draw, corresponding battery power reduction in relation to the total quantity of a remaining dispensable substance. This information may be programmed into the substance cartridge 202. The aerosol test is advantageous because it permits a system that functions without additional hardware, pressure sensors, fluid monitoring, etc., that might be used in various implementations to determine the remaining dispensable substance in a device or cartridge. The aerosol test also has the advantage of permitting measuring the remaining substance without requiring the inhaler apparatus 201 to be held in a particular orientation to accurately gauge the remaining substance. This test permits the measurement to be completed with an insert in the tank in a grid like format so that a mathematical algorithm may be run to determine the remaining volume irrespective of the orientation of the inhaler apparatus 201.

The processor 205 uses this information based on the actual inhalation of the user, to accurately update and monitor the burn rate of a specific user to accurately replenish the supply for the user on time as discussed further herein. For example, the controller 204 includes a counter or timer 207 to determine a quantity such as time of vaporization or duration of inhalation. In particular embodiments, the controller 204 may monitor the duration of vaporization for example by determining the duration and quantity of heating events, by heater 203 configured to heat the substance in the substance cartridge 202 for vaporization of the substance. The controller 204 may be communicably coupled to one or more sensors 208. Sensor(s) 208 may be configured to sense a physical property such as a change in pressure to detect a draw from the vaporization and inhaler apparatus or a change in temperature or other parameter associated with a vapor draw from the vaporization and inhaler apparatus 201. One or more of the quantity and duration of a vapor draw may be analyzed by the processor 205 to determine an estimate of a value associated with a quantity of substance consumed from the substance cartridge 202. The determined quantity value may be stored by the processor, on board the controller 204 and/or on the storage of the substance cartridge 202. The determined quantity values may be aggregated by the processor 205 to estimate a consumption amount and a quantity remaining with respect to an initial quantity.

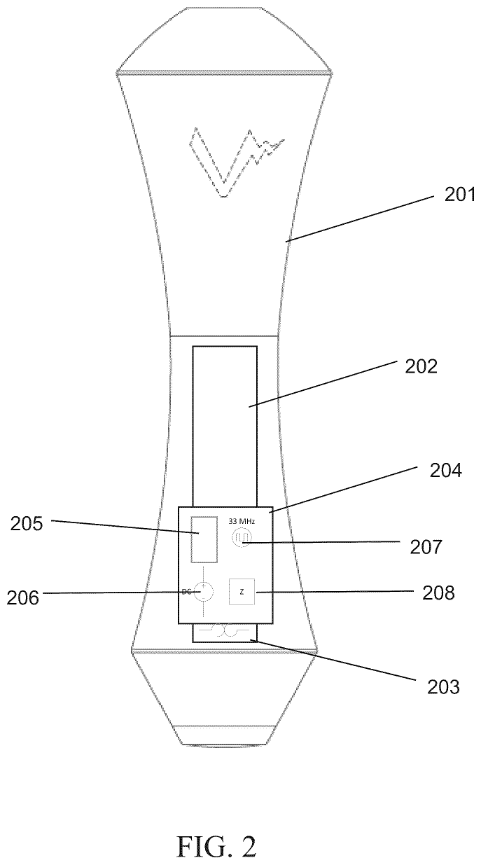

FIG. 3 is a flow diagram illustrating an operational regimen of the vaporization and inhaler apparatus of FIG. 2. At 301, the vaporization and inhaler is activated, for example in response to a user turning the device 201 on, picking the device up, requesting a vapor draw, or inserting a new or different cartridge into the device. At 302, the controller 204 of the device 201 receives a vapor draw request. The vapor draw request may include a requested duration in example embodiments. In various embodiments, the duration of the vapor draw request may be unspecified and the time of the vapor draw may be measured at 303, for example via counter 207. At 303, the duration of the vapor draw may measure a parameter such as the heating duration, which may be controlled for example by the user depressing a button to activate the heater 203 to heat the substance in the substance cartridge 202. In various embodiments, the duration of the vapor draw may measure a parameter such as a pressure change to determine the length of inhalation. At 304, the processor 204 is configured to calculate a vapor draw consumption based on one or more of the measured parameters. In example embodiments, the processor 204 may sense other parameters such as changes in pressure in the cartridge, temperature of the cartridge, changes in weight of the cartridge, or other conditions to determine the vapor draw consumption at 304. Based on the vapor draw quantity, the processor 204 determines the remaining quantity of substance in the substance cartridge 202 at 305. The processor 204 analyzes whether the remaining quantity of the substance in the substance cartridge 202 determined at 305 is at or below a predetermined threshold. The predetermined threshold may be manually programmed by the user or pre-programmed into the processor. The predetermined threshold may be variable based on the user location, which may be determined by a global positioning system in device 201 or other location based system and an estimated delivery time. If the quantity remaining in the substance cartridge 202 is not below the predetermined threshold, the processor 204 will continue to monitor the conditions of future draws. If the quantity remaining in the substance cartridge 202 is below a predetermined threshold, the processor 204 initiates a refill request at 307. In example embodiments, the refill request is sent via a wireless network connection to an ordering system, which may be configured to automatically send and charge the user for the refill or which may simply begin the order and send the user a notification for verification or confirmation of the request. The refill request may include the last location of the user, date and time information, substance identifying information, and user identification or device identification information.

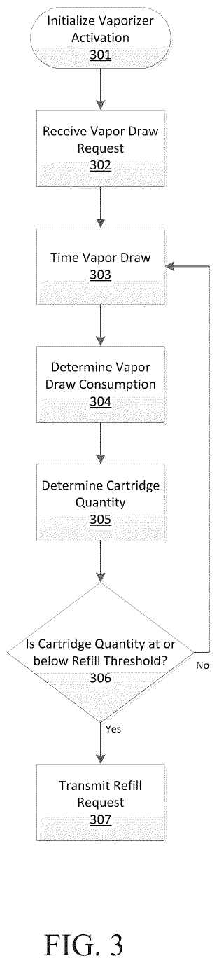

FIG. 4 is a front partially cross-sectional view of a vaporization and inhaler apparatus according another embodiment. A vaporization and inhaler apparatus 401 is configured to allow inhalation of a plurality of different substance loaded into a single cartridge 402. In example embodiments, the vaporization and inhaler apparatus 401 may include vapor draw monitoring systems as discussed in connection with apparatus 201. The cartridge 402 may include a plurality of chambers or silos 403 each filled or fillable with a different substance for consumption by inhaling as disclosed herein. The cartridge 402 may be repositioned to move one or more substance silos into communication with a heater for vaporization of the content within the particular silo. In particular embodiments, the cartridge 402 may be rotatably re-positioned, for example by rotatable actuator 404 controlled by servo-controller 405. In various embodiments, the vaporization and inhaler apparatus 401 may include a single heater configured to heat only a single silo 403 of the cartridge 402 at a time. In other embodiments, the vaporization and inhaler apparatus 401 may include multiple heaters configured to heat more than one silo 403 of the cartridge 402 at a time, based on a user selection.

Repositioning the cartridge 402 for heating a particular silo 403 moves the silo 403 into communication with the heat source and with an output port of the vaporization and inhaler apparatus 401, such that when the selected substance is heated it is consumable by the user. The substance cartridge 401 may include a plurality of substances including, but not limited to caffeine, panax, ginseng, gingko, biloba, bitter orage, cola-nut, guarana, natrum carbonicum, green tea, cocoa extract, cannabis, yerba mate, other vaporizable or inhalable supplements, pharmaceuticals, medicines, waxes, or liquids. The cartridge 402 may be programed with identifying information that indicates what substances are in the cartridge and what silo position the substance is positioned at accordingly. The silos 403 may each have a unique position identifier and each cartridge may have a silo quantity identifier indicating the number and/or position of each silo 403. The information may be stored in a memory device of the cartridge 402 and may be communicably retrieved or sent to the servo controller 405 to control the position of the cartridge in accordance with a user selection.

In particular embodiments, when cartridge 402 rotates, as discussed further herein, it is possible for one sub-cartridge (e.g. silo 403) to have the substance contained therein vaporized at a certain wattage/voltage, while a different sub-cartridge, for example containing a different substance, may be vaporized at a different wattage/voltage. Additionally one sub-cartridge might just dispense (and not vaporize) a substance (pill, powder, liquid, gel) or any combination of the above.

In particular embodiments each of the cartridges 402 and the silos 403, for example a cartridge containing weed, wax, or shatter, operates in a manner similar to an oven. In particular embodiments containing a liquid each of the cartridges 402 and/or the silos 403 use a wick/coil system. In particular embodiments the cartridges 402 and/or the silos 403 may use one or more of an ultrasonic diffuser, a cold air diffuser, an evaporative diffuser, or a heat diffuser. The ultrasonic diffuser uses electronic frequencies to create vibrations that are carried to the surface where oils are floating. The vibrations from the ultrasonic diffuser vaporize the oil and disperse it into the air without using any kind of heat. The cold air diffuser uses room-temperature air to blow the oil into a nebulizer where it is vaporized. The cold air diffuser can diffuse quickly and efficiently. The evaporative diffuser includes a fan that blows air through a pad or filter where the oil sits and vaporizes the oil on the pad. The heat diffuser uses a heat source to disperse the essential oil.

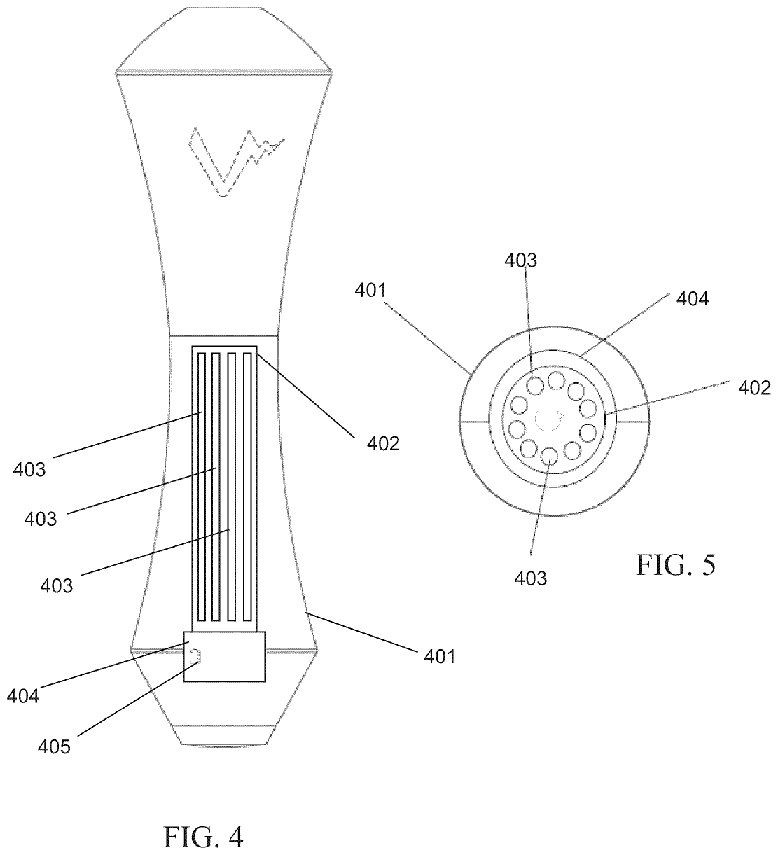

FIG. 5 shows a top cross sectional view of the vaporization and inhaler apparatus 401. As demonstrated in FIG. 5 in accordance with various embodiments, the actuator 404 rotates the cartridge 402 to properly align the selected silo 403 containing a distinct substance for consumption by inhaling to the user preference. In example embodiments, the cartridge may remain stationary and the actuator 404 may reposition the heater for heating the silo 403 and/or may reposition an output port to release the vaporized substance. In various embodiments, the outlet port may be fixed and may be accessible to any vaporized substance of the cartridge 402. In example embodiments, repositioning of at least one of the cartridge and the vaporization heater may be achieved through linear actuation as an alternative to or in addition to rotatable actuation and repositioning.

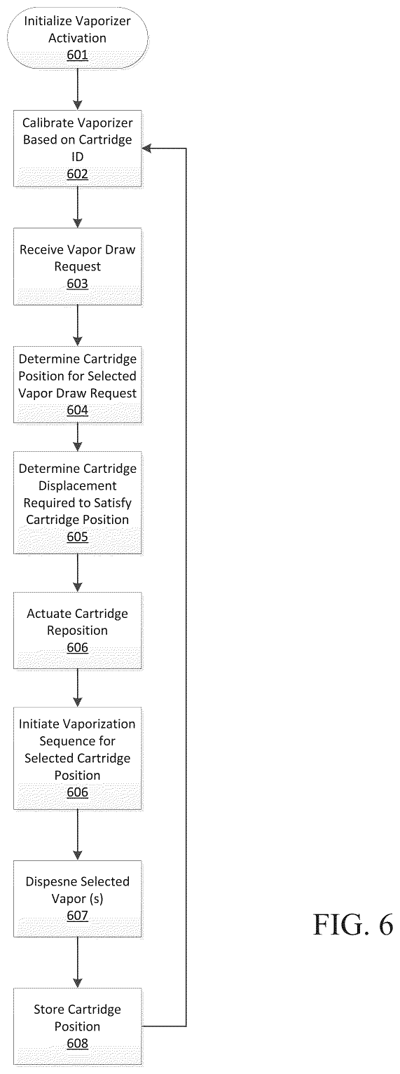

FIG. 6 is a flow diagram illustrating an operational regimen of the vaporization and inhaler apparatus of FIG. 4. At 601, the vaporization and inhaler apparatus 401 is activated, for example in response to a user turning the apparatus 401 on, picking the device up, requesting a vapor draw, or inserting a new or different cartridge 402 into the apparatus 401. At 602, the vaporization and inhaler apparatus 401 is calibrated based on the cartridge inserted in to the apparatus 401 and the information provided by the cartridge 402 regarding the device silos 403, the number of silos, the position of the silos, and the contents of the silos. At 603 the vaporization and inhaler apparatus 401 receives a vapor draw request from a user. The vapor draw request may include an indication of which one or more substance to inhale at a time. If, for example, a cartridge is loaded with two substances that should not be simultaneously mixed, the vaporization and inhaler apparatus 401 may deny the request. If the request is approved for processing, then the servo controller 405 determines the current cartridge position at 604, determines the required cartridge position for vaporization of the appropriate substance at 605, and actuates the actuator 404 at 606 to reposition the cartridge (or heater) for vaporization of the appropriate substance (s). Once the cartridge 402 is correctly positioned with respect to the heater for vaporization of the appropriate one or more substances out of the plurality of substances in the cartridge 402, vaporization is initiated at 606 and the inhalable substance is released at 607. At 608 the instant position and vaporization history associated with the vapor draw are stored so that the next vapor draw position may be appropriately determined.

In certain embodiments, the vaporization system 201 and 401 includes a vaporization control system configured to monitor consumption and/or enable variable product dispensing, for example pursuant to FIGS. 3 and 6. The vaporization control system may include a controller structured to perform certain operations to cause actuation of actuator for dispensing or to cause or prompt automated ordering of refills. The controller may be a single device or a distributed device, and the functions of the controller may be performed by hardware and/or as computer instructions on a non-transient computer readable storage medium.

In certain embodiments, the controller includes one or more modules structured to functionally execute the operations of the controller. In certain embodiments, the controller includes sensor modules configured to measure time lapse, energy consumption, product consumption, rotation position, a change in rotation, linear position, a change in a linear position, product location, product ingredients, or other vaporization system operating parameters or conditions impacting the use, dispensing, or operation of the vaporization system.

The description herein including modules emphasizes the structural independence of the aspects of the controller, and illustrates one grouping of operations and responsibilities of the controller. Other groupings that execute similar overall operations are understood within the scope of the present application. Modules may be implemented in hardware and/or as computer instructions on a non-transient computer readable storage medium, and modules may be distributed across various hardware or computer based components.

Example and non-limiting module implementation elements include sensors providing any value determined herein, sensors providing any value that is a precursor to a value determined herein, datalink and/or network hardware including communication chips, oscillating crystals, communication links, cables, twisted pair wiring, coaxial wiring, shielded wiring, transmitters, receivers, and/or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), and/or digital control elements.

Non-limiting examples of various embodiments are disclosed herein. Features from one embodiments disclosed herein may be combined with features of another embodiment disclosed herein as someone of ordinary skill in the art would understand.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of apparatuses or the components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

While various inventive embodiments have been described and illustrated herein, those of ordinary skill in the art will readily envision a variety of other mechanisms and/or structures for performing the function and/or obtaining the results and/or one or more of the advantages described herein, and each of such variations and/or modifications is deemed to be within the scope of the inventive embodiments described herein. More generally, those skilled in the art will readily appreciate that, unless otherwise noted, any parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way unless otherwise specifically noted. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. An apparatus for vaporization and automatically requesting a cartridge refill, the apparatus comprising:
a cartridge container configured to receive a cartridge including a substance;
a heating element configured to heat the substance of the cartridge to a point of vaporization to generate a vaporized form of the substance;
an outlet port through which the vaporized form of the substance is inhalable by a person;
a wireless transmitter configured to connect the apparatus to one or more networks;
a processor configured to determine quantity of the substance of the cartridge and to monitor a consumption rate of the substance via vaporization; and
wherein if the quantity of the substance remaining in the cartridge is below a predetermined threshold, the processor is configured to initiate a request to order a cartridge to an ordering system via the wireless transmitter.

2. The apparatus of claim 1, wherein the substance comprises cannabis.

3. The apparatus of claim 1, wherein the cartridge comprises an identification code.

4. The apparatus of claim 3, wherein the identification code identifies one or more of the following: substance, flavor identification and expiration information.

5. The apparatus of claim 1, wherein the cartridge comprises a plurality of chambers

6. The apparatus of claim 1, wherein one or more the plurality of chambers comprises a different substance for consumption.

7. The apparatus of claim 1, wherein the apparatus is further configured to position a chamber of the plurality of chambers into communication with the heating element for vaporization.

8. The apparatus of claim 1, wherein the processor is further configured to determine the burn rate based on vapor draw consumption via the outlet port to the user.

9. The apparatus of claim 8, wherein the vapor draw consumption is calculated based at least on one or more of the following parameters: heating duration, length of draw, pressure change.

10. The apparatus of claim 8, wherein the vapor draw consumption is calculated based at least one or more of the following parameters: changes in pressure in cartridge, temperature of cartridge, change in weight of cartridge.

11. The apparatus of claim 1, wherein the ordering system is further configured to automatically send and charge the user for the refill.

12. The apparatus of claim 1, wherein the cartridge is one of programmable or comprises memory communicably coupled to the processor to provide historical information such as one or more of the following: number of prior uses, initial quantity and remaining quantity.

13. The apparatus of claim 1, wherein the processor is further configured to vary the consumption rate based at least on power of the apparatus, substance in the cartridge or time of use.

14. The apparatus of claim 1, wherein the predetermined threshold is configured to be adjustable based on one or more of the following: user habits, user location(s), and estimated time of delivery of order.

15. The apparatus of claim 1, wherein the apparatus further comprises a location based system to determine a location of the user.

16. The apparatus of claim 1, wherein the request comprises one or more of the following: a location of the user, date and time information, substance identifying information, user identification and apparatus identification.

17. An apparatus for vaporization of different substances using a cartridge with a plurality of chambers, the apparatus comprising:
a cartridge container configured to receive a cartridge having a plurality of chambers, each chamber of the plurality of chambers filled with a different substance for consumption;
an actuator configured to rotate the cartridge to align a selected chamber for vaporization;
a heating element configured to heat a substance of a selected chamber to a point of vaporization to generate a vaporized form of the substance of the selected chamber;
an outlet port through which the vaporized form of the substance of the selected chamber is inhalable by a user.

18. The apparatus of claim 17, wherein the different substance within each of the chambers comprises one or more of the following: caffeine, panax, ginseng, gingko, biloba, bitter orage, cola-nut, guarana, natrum carbonicum, green tea, cocoa extract, cannabis and yerba mate.

19. The apparatus of claim 17, wherein the different substance within each of the chambers comprises one or more of the following: supplements, pharmaceuticals, medicines, waxes, or liquids.

20. The apparatus of claim 17, wherein the actuator is further configured to rotate a chamber of the plurality of chambers of the cartridge into communication with the heating element.
